(19) United States
(12) Patent Application Publication
(10) Pub. No.: US 2023/0321375 A1
(43) Pub. Date: Oct. 12, 2023

(54) RESPIRATORY THERAPY DEVICE

(71) Applicant: CEGLA Medizintechnik GmbH & Co. KG, Montabaur (DE)

(72) Inventors: Ulrich Hartmann Cegla, Montabaur (DE); Andrea Ebinger, Montabaur (DE)

(21) Appl. No.: 18/021,056

(22) PCT Filed: May 3, 2021
(86) PCT No.: PCT/EP2021/061531

§ 371 Date: Feb. 13, 2023

(30) Foreign Application Priority Data
Aug. 11, 2020 (EP) 20190395.2

(51) Int. Cl. A61M 16/00; A61M 16/20

(52) U.S. Cl. CPC A61M 16/0006; A61M 16/20; A61M 16/0488

(57) ABSTRACT

The invention relates to a respiratory therapy device (1) for treating the musculature of respiratory tracts, lung functions and/or mucous deposits in the lungs, nose and/or throat of a patient (2), consisting of a hollow body (3) in which at least one through-channel (4, 5) is provided, a mouthpiece or nosepiece (6) which is inserted into the hollow body (3) and which is connected to the patient (2) during the duration of the treatment by the respiratory therapy device (1), and consisting of at least one vibration body (11, 12) which is arranged in the interior of the hollow body (3) and which is caused to vibrate in an oscillating manner by the inhalation and/ or exhalation of the patient (2), wherein an oscillating, resistive and/or threshold-typical air pressure vibration occurs, the individual resistances during the inhalation and exhalation being adjustable specific to the patient and a modification or repositioning of the respiratory therapy device (1) not being required. This is solved in that a retaining body (13) is inserted into the hollow body (3), into which retaining body at least one opening (14) is integrated, in that a freely oscillating tongue (15) is fixed to the hollow body (13) in the region of the opening (14), by means of which tongue the opening (14) of the retaining body (13) is closed or opened in regions according to the air flows (7, 8) prevailing in the interior of the hollow body (3), in that the end face (9) of the hollow body (3) opposite the mouthpiece (6) is open, in that a stenosis (18) is inserted into said end face (9), into the shell surface (19) of which stenosis at least two passage openings (20) are provided through which the air flows (7, 8) flow into the interior of the hollow body (3) or vice versa, in that the passage openings (20) of the stenosis (18) have different cross-sectional areas than air passages for the air flows (7, 8) and that the stenosis (18) is rotatably mounted in the hollow body (3), such that one of the passage openings (20) can be selected or adjusted as an air passage by rotating the stenosis (18).

### REFERENCE TO PENDING PRIOR PATENT APPLICATIONS

This patent application is a 371 national stage entry of pending prior International (PCT) Patent Application No. PCT/EP2021/061531, filed 3 May 2021 by Cegla Medizintechnik GmbH & Co. KG for RESPIRATORY THERAPY INSTRUMENT, which patent application, in turn, claims benefit of European Patent Application No. 20190395.2, filed 11 Aug. 2020.

The two (2) above-identified patent applications are hereby incorporated by reference.

### FIELD OF THE INVENTION

The invention relates to a respiratory therapy device for treating the musculature of respiratory tracts, lung functions, and/or for clearing mucous in the lungs, nose, and/or throat of a patient according to the preamble of Claim 1.

### BACKGROUND OF THE INVENTION

Such a respiratory therapy device is described in EP 3 251 718 B1, for example. A flexible body in the form of a hose is fastened to a mouthpiece inside a tube piece. The tube piece is curved, so that the hose has a curvature and consequently, a cross-sectional reduction in the region of the curvature. As soon as a patient inhales or exhales respiratory air through the mouthpiece, the ambient air or the respiratory air is drawn in or discharged, respectively, through the hose, so that the hose is set into oscillation or vibration. This vibration results in oscillating air pressure fluctuations in the mouth, nose, and/or lungs of the patient, resulting in the musculature of the respiratory tracts as well as the clearing of mucous in these areas results.

A ventilation device has become known from EP 3 620 195 A1. The ventilation device is used to treat an artificially ventilated patient, and at the same time, to clear mucous in the bronchial tubes and to train the respiratory musculature. This device is made up of a respiratory therapy device, for example of the species referenced above.

In practice, such respiratory therapy devices have proven to be valuable for decades, and are successfully used to treat patients. Nevertheless, it has turned out to be disadvantageous that the hose that is used has a very short service life on the one hand, and on the other hand may become obstructed even after a few breaths. In addition, aerosols, mucous, or bacteria or viruses deposit inside the hose and must be removed after each treatment. After a few treatment procedures, it is thus necessary to laboriously remove the hose from the tube piece and the mouthpiece and place them, for example, in a microwave or some other medical device that is suitable for sterilization in order to remove such pathogens.

The respiratory musculature, as with any musculature, may be trained. In muscle training, medical professionals make a distinction between strength training and endurance training. The devices thus far for training the respiratory musculature are either resistive devices (resistance-generating devices) or so-called threshold devices. In threshold devices, a spring valve opens when a certain pressure is overcome, and unblocks an opening.

When resistive respiratory musculature trainers are used, a stenosis is generally provided upstream from the respiration.

A certain resistance must be overcome for training the musculature. In order to carry out a comparable training, this resistance understandably must be identical for each training.

All devices thus far on the market are largely intended for inhalation or exhalation, and have only static pressure generation (positive inspiratory pressure (PIP)).

A respiratory training device that dilates the bronchial tubes and clears or liquefies mucous trapped therein via resistive oscillating positive inspiratory pressure (OPIP) and in a threshold-driven manner as of yet does not exist in conjunction with the respiratory musculature training.

Further respiratory therapy devices of this type are known from US 10,004,872 B1, EP 0 262 239, US 2019/0201743 A1, or WO 2019/070804 A1, for example. The respiratory therapy devices described therein share the feature that a valve in the form of a plate or tongue that swings up is situated in a flow channel; pressure fluctuations arise or are formed by the plate or tongue as a function of the generated air pressures, i.e., the inhalation or exhalation pressure that is generated by a patient. The drawn-in or discharged air flows perpendicularly onto a surface of the tongue or plate that is lifted from the retaining plate by the prevailing pressure of the air, so that the air may flow through the air gap between the tongue and the top side of the retaining plate.

In such respiratory therapy devices, it is considered disadvantageous that the drawn-in or discharged air acts on the tongue perpendicularly, since this results in no vibration of the tongue. Namely, the tongue can only be lifted or closed. This corresponds to a known valve function. Oscillation of the tongue does not result from this arrangement and the associated air flow.

Furthermore, in the respiratory therapy devices that have become known, the air volume that is drawn or discharged into the through channel of the particular hollow body is not individually adjustable to the specific patient. Rather, a constant predefined air volume passes into the through channel of the hollow body or is discharged from same. This volume fraction of the drawn-in or discharged air corresponds to the lung volume of the particular patient. However, since numerous different patients and illnesses or training purposes are to be handled using a respiratory therapy device, the adjustment options for the known respiratory therapy devices are very limited.

### SUMMARY OF THE INVENTION

The object of the invention, therefore, is to refine a respiratory therapy device of the species stated at the outset, in such a way that that an oscillating, resistive, and/or threshold-typical air pressure oscillation arises, in which the individual resistances during the inhalation and exhalation are individually adjustable to the specific patient without the need to modify or convert the respiratory therapy device. Depending on the length, weight, stiffness, and position of the tongue as well as the design or the arrangement of the cavity, the training load may additionally be individually adapted.

This object is achieved by the features of the characterizing part of Claim 1.

Further advantageous refinements of the invention result from the subclaims.

As the result of inserting a retaining body, into which at least one opening is incorporated, into the hollow body, fastening a freely oscillating tongue, made of a metallic material, to the hollow body in the region of the opening, the opening of the retaining body being closed or opened up in areas via the tongue as a function of the air flows prevailing in the interior of the hollow body, the end face of the hollow body opposite from the mouthpiece being open, and inserting a stenosis into the end face, at least two through openings being provided in the shell surface of the stenosis through which ambient air flows into, or conversely, flows out of, the interior of the hollow body, the through openings of the stenosis having different cross-sectional areas than air passages for the air flows, and rotatably supporting the stenosis in the hollow body in such a way that one of the through openings is selectable or adjustable as an air passage by rotating the stenosis, the intent is to achieve regulation, individually adjustable to each patient, of the pressure state in the interior of the hollow body. Via this mechanism, oscillating pressure fluctuations of various types are generated which are effective for bronchial dilation, stabilization, and clearance of mucous. Such a respiratory therapy device is thus usable for individual medical respiratory musculature training purposes, in which an oscillating, resistive, or threshold-driven bronchial tube dilation is provided during inhalation and/or exhalation of the respiratory air.

The subject matter of the invention uses tongues for generating acoustically perceivable oscillations, which due their differing frequencies allow inspiration to be distinguished from expiration. Depending on the conditions and the location, a different level of defined pressure or flow is necessary to set the tongues into oscillation. Via upstream connection of different stenoses, the pressure or flow at which the tongue begins to oscillate may be varied and adjusted. An audible tone occurs when the desired pressure or flow is reached. When the device is utilized as a muscle trainer, the occurrence of such a tone indicates that the adjustable “minimum counterforce” for the training has been reached.

The resulting different tones are important with regard to their sound intensity, their duration, and their temporal relationship with the exhalation for evaluation or guidance during the therapy or the training.

In particular when the oscillating body is formed from a retaining body having a cuboidal box profile, at which the openings for the passage of air are incorporated into the two lateral side walls extending in parallel to the vertical, the tongues may be set into oscillation solely by the inhalation or exhalation activity of the patient, without influence by the prevailing force of gravity. In addition, the air does not flow perpendicularly onto the tongues, but instead sweeps between the free ends of the tongues and the top or bottom side of the retaining body in the region of the openings, resulting in vibration at the free ends of the tongues, which generates the desired and necessary oscillation of the air pressures in the hollow body.

These vibrations also generate acoustics, since the particular tongue is to be regarded as a sounding body. Solely this vibration of the tongue and the associated sound serve to identify the inhalation or exhalation behavior for the patient. Namely, if the generated tone is too low or too high, or the sound intensity is too low, the patient recognizes immediately, i.e., during the inhalation or exhalation process, that these breathing activities do not meet the desired therapeutic or training purpose, and may thus promptly adapt or change his/her lung activity.

In addition, the drawn-in ambient air may be variably modified via the through openings that are incorporated into the stenoses, since the cross-sectional areas of the particular through opening have different sizes at the stenosis, and since the stenosis is rotatably supported in the hollow body, so that the patient, even during use of the respiratory therapy device, may individually adjust the corresponding drawn-in or discharged air volume by rotating the stenosis. Due to the generated vibrations and the corresponding air flows, the sound produced at the tongues also allows the patient to immediately check the extent to which the selected settings meet the desired therapeutic or training purpose.

Various stenoses are available which may be mounted quickly and easily.

Since the respiratory tract resistance is defined as the change in pressure divided by the change in flow, there must be an identification of reaching the desired pressure that is recognizable by the patient, since otherwise, different pressures are present due to different flows for an identical stenosis, resulting in a different training load.

The start of oscillation of a reed is a function of its stiffness and mass. For a defined flow, identical reeds have the identical start of oscillation. For the same flow, the different diameters of stenoses result in different pressures. Combining a reed with a downstream connection of a stenosis having a defined diameter thus generates a reproducible, defined pressure integral upon the start of oscillation of the tongue. This may be acoustically perceived and thus recorded. The respiratory musculature must work against this pressure integral, and via same is trained in a defined and reproducible manner.

The force necessary for setting the tongue into oscillation and generating a tone is a function of the stiffness and the length of the tongue, and of the installation angle. When this force is known, for a predefined stenosis the resistance that is achieved may be exactly determined. Since the stiffness of the tongue does not change, the pressure at which it starts to oscillate is always identical. The “critical pressure” and therefore the overcome resistance may thus be accurately determined for all stenoses.

In addition to changing the installation angle, the selection of the different stenoses for which the tongue begins to oscillate or the through opening of the stenosis is open may also result in a different pressure.

The resulting pressure fluctuations due to the oscillations of the tongue dilate the bronchial tubes, and bronchial mucous, since it is thixotropic, is sheared from the walls and liquefied.

When a single reed is used as a “pressure generator and indicator” without stenosis, the oscillation results in a pressure pattern that equals the dynamic zero-to-peak, peak-to-zero (PEP); i.e., the pressure increases from 0 to a maximum and then immediately drops back to 0. In addition to the length of the reed, its shape is also responsible for a certain pressure pattern. The longer and heavier or stiffer the tongue, the higher the initial pressure. The tongue must have enough space in the structure so that the clearance distance can be maintained in order for the tongue to swing up.

The longer the tongue, the greater the distance it requires. Therefore, long and heavy reeds are used for the dynamic pressure form. The resulting pressure form is necessary for moving the bronchial mucous, and for the shearing and liquefying.

If instead of the single reed, a free reed is used which likewise indicates reaching a certain pressure during tone generation, the pressure fluctuation pattern here corresponds to a combined PEP. The pressure fluctuation is made up of a permanently positive pressure.

By using two different oscillating bodies, it is also possible to distinguish between inspiration and expiration solely acoustically. These distinctions may be recorded, evaluated, and documented with regard to frequency, length, and OPIP or OPEP application (different harmonics), using an app.

An additional variable stenosis may be connected between the mouthpiece and the tubes leading to the tongues.

By use of the arrangement according to the invention of oscillating bodies, having different designs, in a hollow body that is also used, for example, with an inhalation device or in a hose system of a ventilation device, any state of a patient may thus be taken into account. For artificially respirated patients, the respiratory therapy device described according to the invention may be connected to and disconnected from the ventilation circuit. The bronchial mucous may thus be cleared and liquefied, and the respiratory musculature may be easily trained.

### BRIEF DESCRIPTION OF THE DRAWINGS

Eight embodiment variants of a respiratory therapy device are illustrated in the figures and explained in greater detail below. In the figures:

FIG. 1 shows a first exemplary embodiment of a respiratory therapy device made up of a hollow body and a mouthpiece fastened thereto, and via which a patient draws in respiratory air from the surroundings, the free end of the hollow body being provided with a stenosis into which three through openings having different opening cross sections are incorporated and reduced in size, a first oscillating body being situated in a through channel of the hollow body, in a side view,

FIG. 2a shows an enlargement of the first oscillating body in the form of a retaining plate into which a rectangular opening is incorporated, and which is covered by a tongue, in the lifted state according to FIG. 1,

FIG. 2b shows the retaining plate according to FIG. 2b with a lowered tongue via which the exit opening is closed,

FIG. 2c shows an elongation of the opening with a tongue that penetrates the opening in both directions,

FIG. 3a shows a second exemplary embodiment of a respiratory therapy device that may be used for both inhalation and exhalation, having two hollow bodies that open into one another, and whose respective free end faces are closed by one of the stenoses, and having three oscillating bodies, situated in the interior of the hollow body, in the form of a retaining plate with a tongue or a valve, during the inhalation process,

FIG. 3b shows the respiratory therapy device according to FIG. 3a during the exhalation process,

FIG. 4 shows a third exemplary embodiment of a respiratory therapy device in which the oscillating bodies are situated at an angle to the respective axis of symmetry of the hollow body or extend orthogonally with respect to same,

FIG. 5 shows the respiratory therapy device according to FIG. 1, to which sensors and microphones that communicate with an external device are attached to the oscillating body of the respiratory therapy device,

FIG. 6a shows a fourth exemplary embodiment of a respiratory therapy device, in whose hollow body a partition wall is provided which divides the hollow body into two through channels through which the respiratory air flows during inhalation, at least one of the two oscillating bodies being installed in each through channel,

FIG. 6b shows the respiratory therapy device according to FIG. 6a, via which the exhaled respiratory air is pushed back through the second through channel and into the surroundings,

FIG. 7 shows a fifth exemplary embodiment of a respiratory therapy device in one refinement of the embodiment version according to FIGS. 6a and 6b, with two oscillating bodies provided in the respective through channel,

FIG. 8 shows a sixth exemplary embodiment of a respiratory therapy device, with two floating bodies situated in the respective through channels of the respiratory therapy device according to FIG. 6a, in an orthogonal or oblique arrangement,

FIG. 9 shows a seventh exemplary embodiment of a respiratory therapy device according to FIG. 1, in which a cuboidal box profile is inserted into the hollow body as a retaining body for the tongues, in a top view,

FIG. 10 shows the respiratory therapy device according to FIG. 9 with two air flows illustrating the inhalation and exhalation processes as well as the oscillation behavior of the tongues,

FIG. 11a shows one of the stenoses inserted into the hollow body of the respiratory therapy device according to FIGS. 1 through 10, with air cross-sectional areas having different sizes, as a first alternative,

FIG. 11b shows a second alternative for a stenosis according to FIG. 11a,

FIGS. 12a through 12c show a partial cross section of the retaining body according to FIGS. 9 and 10, with different oscillation behaviors of the tongues mounted thereon,

FIG. 13 shows an air flow diagram for different stenoses and tongues according to one of the respiratory therapy devices according to FIGS. 1 through 10,

FIG. 14 shows one of the respiratory therapy devices according to FIGS. 1 through 10, with a Y-shaped adapter which divides the open end face of the hollow body into two air inlet or exit openings, and

FIG. 15 shows one of the respiratory therapy devices according to FIGS. 1 through 10 in a nasal application, with a mask connected thereto.

### DETAILED DESCRIPTION OF THE PREFERRED EMBODIMENTS

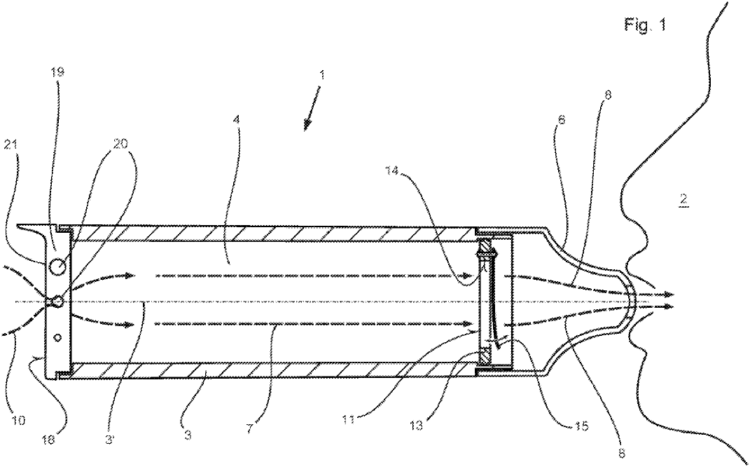

FIG. 1 depicts a respiratory therapy device 1 via which a patient 2 may improve his/her respiratory musculature for clearing mucous in the bronchial tubes, and/or his/her lung function. The respiratory therapy device 1 is made up of a hollow body 3 that is designed as a tube piece, for example. Provided at one of the free ends of the hollow body 3 is a mouthpiece 6 that may be connected to the patient 2, so that the patient 2 by means of his/her respiratory musculature may draw in ambient air 10 through the mouthpiece 6.

The end face of the hollow body 3 oppositely situated from the mouthpiece 6 is closed by a stenosis 18. Three through openings 20 having differently sized diameters or cross-sectional areas are incorporated into the shell surface 19 of the stenosis 18. As soon as the patient 2 draws in ambient air 10 through the mouthpiece 6, the air flows through the through openings 20 and into the interior of the hollow body 3, which is thus used as a through channel 4 for the drawn-in ambient air 10. The air flows that arise in the interior of the hollow body 3 are denoted by reference numerals 7 and 8, with reference numeral 7 representing the ambient air to be inhaled, and reference numeral 8 representing the exhaled air of the patient. Due to the number of through openings 20 and their diameters, the patient 2 already requires increased breathing power to draw the ambient air 7 into the through channel 4 at all.

Since the respiratory air is to build up vibrating or oscillating pressures to the greatest extent possible, a first oscillating body 11 is situated in the through channel 4, directly upstream from the mouthpiece 6.

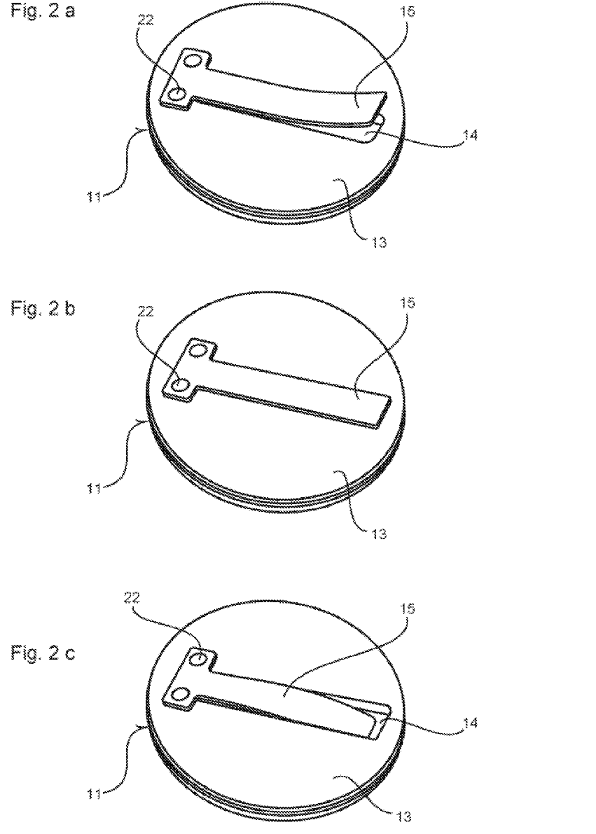

According to FIG. 2a, the first oscillating body 11 includes a plate-shaped retaining body 13 into which a rectangular opening 14 is incorporated. In the unactuated state, the opening 14 is closed in areas by a tongue 15. A free end of the tongue 15 is fastened to the retaining body 13 by means of two screws 22. The outer contour of the retaining body 13 is adapted to the inner contour of the through channel 4 of the hollow body 3, so that the drawn-in respiratory air 7 can flow only through the opening 14 in the direction of the mouthpiece 6, since the shell surface of the retaining body 13 rests against the inner wall of the through channel 4 in an airtight manner. The tongue 15 accordingly swings in the direction of the mouthpiece 6 as soon as the patient 2 draws respiratory air 7 through the mouthpiece 6. At the free end face situated opposite from the screws 22, the tongue 15 is thus lifted by the inhalation pressure applied by the patient 2, and the exit opening 14 is opened up in areas.

As soon as the inhalation process is ended by the patient 2, according to FIG. 2b the tongue 15 springs back onto the top side of the retaining body 13, and the opening 14 is consequently completely closed so that it is sealed airtight. The vibrating or oscillating air pressure fluctuations generated by the tongue 15 are thus a function of the flexural rigidity of the tongue 15 and the mass of the tongue 15, and also of the dimensions of the diameter of the stenosis 18 or of the opening 14.

FIG. 2c shows that the opening 14 may be elongated so that the length of the tongue 15 is smaller than the length of the opening 14. This means that an air gap is formed between the free end of the tongue 15 and the inner side of the opening 14, and the tongue 15 may swing or dip through the opening 14 in both directions. As a result, the tongue 15 swings through the opening 14, and with such a design of the oscillating body 11, the respiratory therapy device 1 according to FIG. 1 may be used for both inhalation and exhalation.

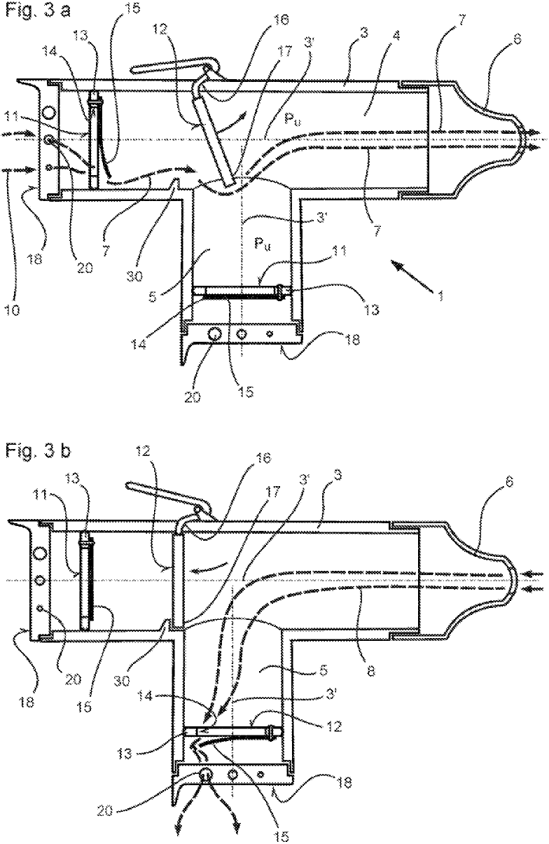

FIGS. 3a and 3b show another type of design of the respiratory therapy device 1, which now has two interconnected hollow bodies 3 whose respective axes of symmetry, denoted by reference numeral 3′, extend perpendicularly to one another. The respective free end faces of the two hollow bodies 3 are closed by the stenosis 18. Two oscillating bodies 11 and 12 are situated in the first through channel 4.

The first oscillating body 11 includes the retaining body 13 with the opening 14, which is closed or partially opened up by the tongue 15 according to FIGS. 2a and 2b. In addition, a second oscillating body 12 whose first end face is swivelably connected to the hollow body 3 via a swivel joint 16 is situated in the through channel 4. When the patient 2 draws ambient air 7 in through the stenosis 18 into the through channel 4, the oppositely situated free end of the oscillating body 12, denoted by reference numeral 17, may lift off from a stop 30 that is integrally formed on the inner side of the hollow body 3. The second oscillating body 11 acts as a type of valve.

In the process, the tongue 15 of the first oscillating body 11 lifts off from the opening 14. A negative pressure denoted by reference symbol Pu develops in the through channel 4. Since the two through channels 4 and 5 are connected to one another, the negative pressure Pu develops in the second through channel 5, as the result of which the tongue 15 of the first oscillating body 11 is pressed onto the retaining body 13 in order to close the opening 14. Namely, the tongue 15 is situated on the side of the retaining body 13 facing away from the first through channel 4, so that the negative pressure Pu pulls the tongue 15 onto the retaining body 13.

During the exhalation according to FIG. 3b, the two oscillating bodies 11 and 12 in the first through channel 4 are closed, since the valve 16 is pressed against the stop 30 by the respiratory air 8 during exhalation; the opening 14 likewise closes, since the tongue 15 is pressed onto the retaining body 13 due to the prevailing pretensioning force of the tongue 15, and thus rests thereon. The exhaled respiratory air 8 accordingly flows through the mouthpiece 6 and into the first through channel 4, and then into the second through channel 5, so that the respiratory air 8 impinges on the tongue 15 of the oscillating body 11 situated in the through channel 5, and in areas lifts or presses the tongue 15 from the opening 14. The respiratory air 8 subsequently flows through the through opening 20 of the stenosis 18, situated in the free end face of the second hollow body 3, and into the surroundings.

The respiratory therapy device 1 according to FIGS. 3a and 3b may therefore be used for both inhalation and exhalation. Due to the two differently selectable stenoses 18 for inhalation and exhalation, the training load for these two breathing maneuvers may be adjusted individually and separately.

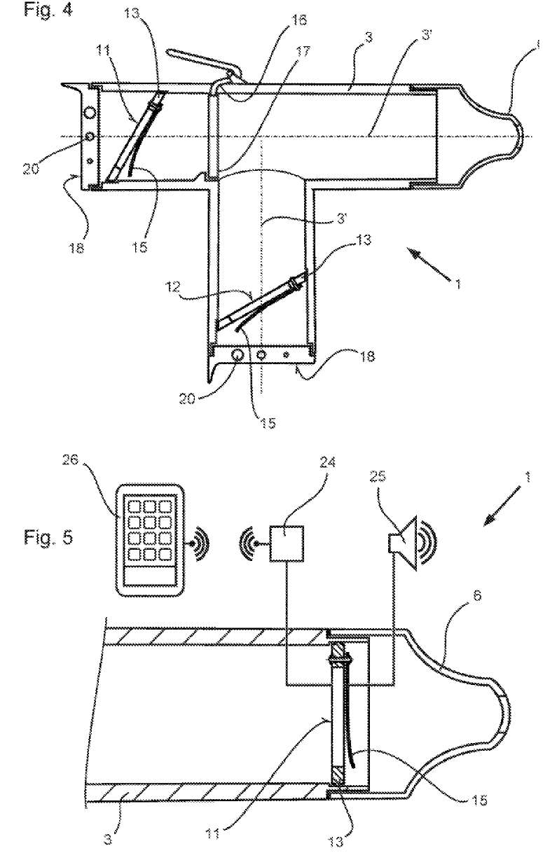

FIG. 4 illustrates another type of arrangement of the oscillating bodies 11 in the two hollow bodies 3. According to FIGS. 3a and 3b, the oscillating bodies 11 extend perpendicularly to the axis of symmetry 3′ of the respective hollow body 3, and in FIG. 4 it is apparent that the oscillating bodies 11 are situated at a predefined angle, i.e., inclined with respect to the respective axis of symmetry 3′.

According to FIG. 5, the oscillating body 11 is to be utilized as both an acoustic and an electronic transmission unit. Speakers 25 or electrical sensors 24 are therefore provided at the tongue 15, for example. The speakers 25 transmit a tone, so that during use of the respiratory trainer (inspiration) or respiratory therapy device (expiration) 1, the patient 2 can acoustically perceive whether his/her respiratory musculature sufficiently sets the tongue 15 into oscillation, and how long he/she can maintain the oscillating resistance, i.e., the predefined training load.

The sensors 24 communicate with an external device 26 via a WLAN connection, for example. The device 26 may record the period of use, the frequencies of the tongue 15 that are used, and the number of breathing exercises of the patient 2, so that they may be read out and evaluated by a medical practitioner, for example, after a certain period of time.

If two different tongues 15 are installed, a distinction may be directly made between the training of the inspiratory musculature, including the corresponding training load, and for exhalation, the duration, including the resistive and threshold-driven level of therapy, and may be recorded. A conventional mobile phone may also be used for these recordings via an app, so that the speakers and microphones could be superfluous.

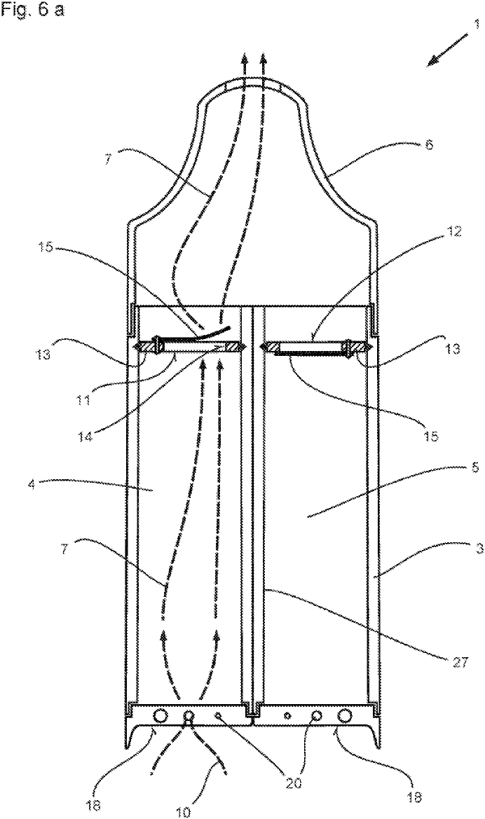

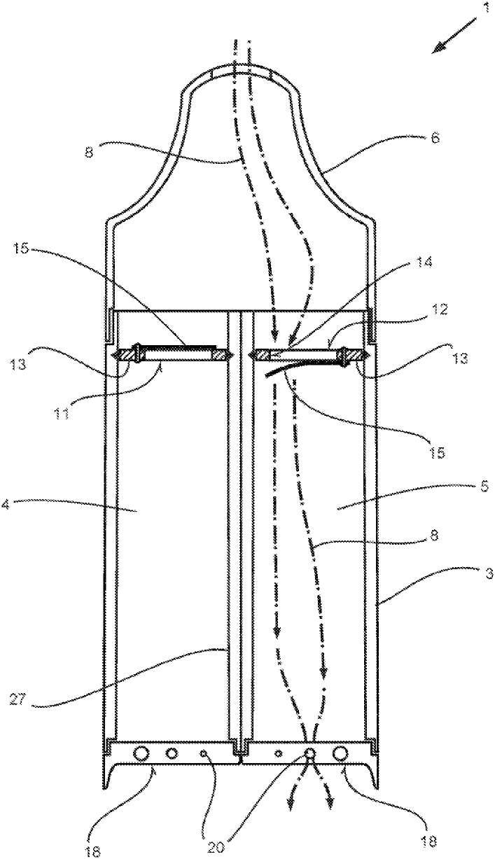

In FIGS. 6a and 6b, the respiratory trainer or the respiratory therapy device 1 includes a hollow body 3 in which a partition wall 27 is provided for forming two through channels 4 and 5 that are separated from one another in an airtight manner. The respective free end faces of the hollow body 3 are partially closed by the stenosis 18. One of the oscillating bodies 11 is situated in each of the through channels 4 and 5. According to FIG. 6a, the oscillating body 11 provided in the through channel 4 is opened as soon as the patient draws in respiratory air 7 through the through channel 4, since the tongue 15 is pressed in the direction of the mouthpiece 6 by the inhaled respiratory air 7. The tongue 15 is situated on the top side of the retaining plate 13 facing the mouthpiece 6.

According to FIG. 6b, the tongue 15 in the through channel 5 opens as soon as the patient 2 exhales, since the respiratory air 8 initially presses the tongue 15 of the oscillating body 11 in the through channel 4 onto the retaining body 13, and the through channel 4 is subsequently closed and the respiratory air 8 presses on the tongue 15 of the oscillating body 11 situated in the through channel 5. The tongue 15 is situated on the top side of the retaining body 13 facing away from the mouthpiece 6.

FIG. 7 depicts a combination of a first oscillating body 11 with a free reed 15 and a second oscillating body 12. During inhalation, the through channel 4 is opened by means of the oscillating body 11 and the oscillating body 12, and during exhalation these are closed by the respiratory air 8, so that the respiratory air 8 presses on the oscillating body 12 in the through channel 5.

It is possible to use a combination of an inhalation device/nebulizer 36 with the second oscillating body 12. The through channel 4 is opened by the oscillating body 12 during inhalation, and during exhalation is closed by the respiratory air 8, so that the respiratory air 8 presses on the oscillating body 12 in the through channel 5.

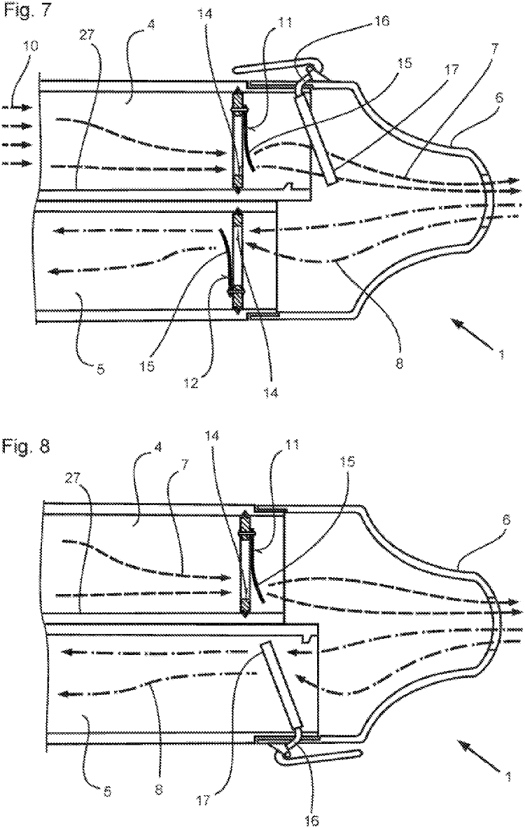

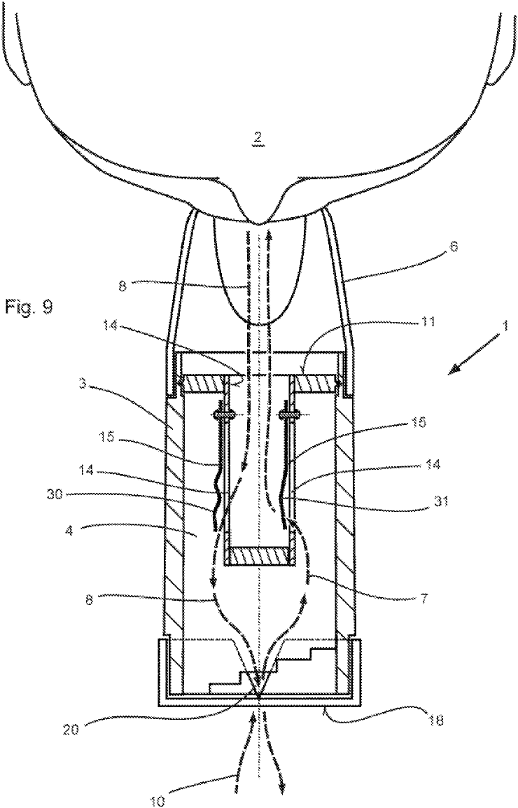

FIG. 8 shows that the through channel 4 is closed by an oscillating body 11, and the through channel 5 is closed by an oscillating body 12. These combinations may optionally be correspondingly exchanged, and the orientation of the oscillating bodies 11 and 12 may extend perpendicularly or at an angle to the hollow body 3 relative to the axis of symmetry 3′ of the hollow body.

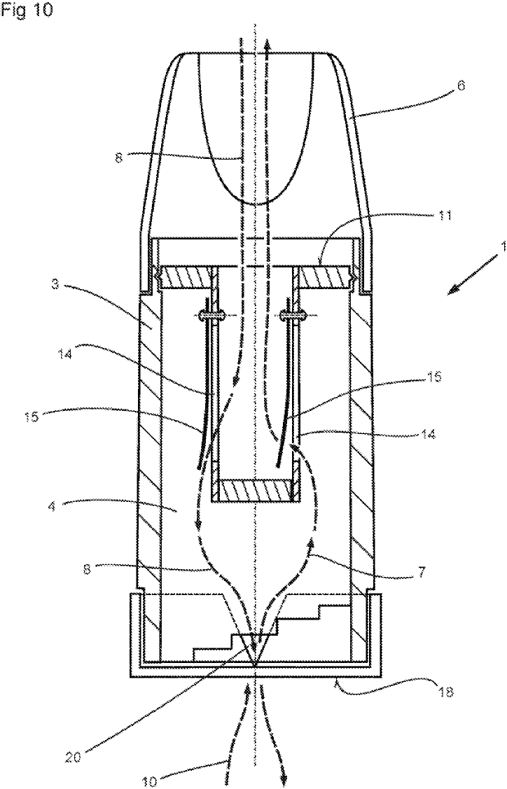

FIGS. 9 and 10 show a further alternative embodiment of the respiratory therapy device 1. The oscillating body 11 is firstly made up of the retaining body 13 having a T-shaped cross section. The free ends of the retaining body 13 are fastened to the inner side of the hollow body 3, which encloses one of the openings 14. This opening 14 accordingly extends oppositely from the mouthpiece/nosepiece 6 or opens into same, so that the air flows 7, 8 sweep essentially perpendicularly through these openings.

In addition, the respiratory therapy device 1 is provided with a cuboidal box profile as an integral part of the retaining body 13. The cuboidal box profile accordingly encloses a cavity that communicates with the opening 14 or opens into same. Two oppositely situated side faces of the box profile 13 have two openings 14 at which the tongues 15 are exteriorly or interiorly mounted. When the respiratory therapy device 1 is used, the two side faces of the retaining body 13 extend in parallel to the vertical, so that intrinsic weight forces of the tongues 15 do not influence their oscillation behavior; rather, the oscillation behavior of the tongues 15 is influenced solely by the generated air pressures, air fluctuations due to the through openings 20 provided at the stenosis 18, and the material characteristics of the tongues 15 or the geometry in the opening 14. The tongues 15 thus move in a vertical plane, and may be set into corresponding vibrations or oscillations during both inhalation and exhalation of the patient 2. The flow behavior in the interior of the hollow body 3 and in the region of the openings 14 and the tongues 15 is schematically illustrated by reference numerals 10, 7, and 8. Thus, when the patient 2 inhales, the ambient air 10 is drawn in through one of the through openings 20 of the stenosis 18 and into the through channel 4 of the hollow body 3, and the interiorly mounted tongue 15 swings up, so that the inhaled ambient air 7 flows through the opening 14 and the air gap between the tongue 15 and the retaining body 13 and through the opening 14 facing the patient 2, and may be inhaled by the patient. During exhalation, the respiratory air 8 flows into the interior of the box profile of the retaining body 13 and presses against the interiorly mounted tongue 15 so that its opening 14 is closed; in contrast, the exteriorly deflected tongue 15 swings outwardly, and the respiratory air 8 passes through the air gap, opened up by the tongue, into the through channel 4 of the hollow body 3, and from there, through the through opening 20 of the stenosis 18 to the outside.

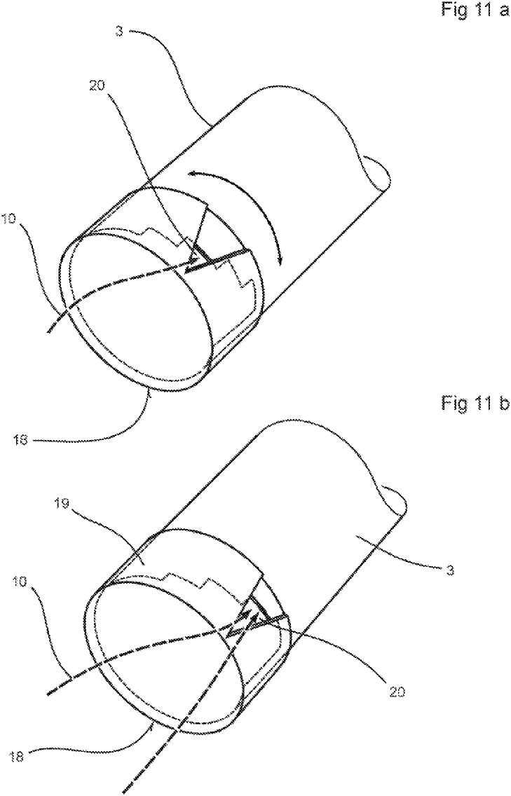

FIGS. 11a and 11b illustrate two different exemplary embodiments of one of the common stenoses 18, having different sizes of through openings 20. The respective stenosis 18 is rotatably supported in the hollow body 3, and its shell surface 19 has four through openings 20 having different designs, which by rotating the stenosis 18 relative to the hollow body 3 may be positioned in such a way that the corresponding inlet opening is decreased or increased in size. As a result of appropriately selecting the cross-sectional area of the particular through opening 20, the patient 20 must draw the ambient air 10 into the through channel 4 of the hollow body 3 with more or less suction pressure, or must press out the respiratory air 8 from the through channel 4 into the open air. In addition, the appropriately adjusted cross-sectional area of the through openings 20 has a significant influence on the oscillation behavior of the particular tongue 15, since the oscillation behavior of the particular tongue 15 changes at a higher internal pressure in the through channel 4.

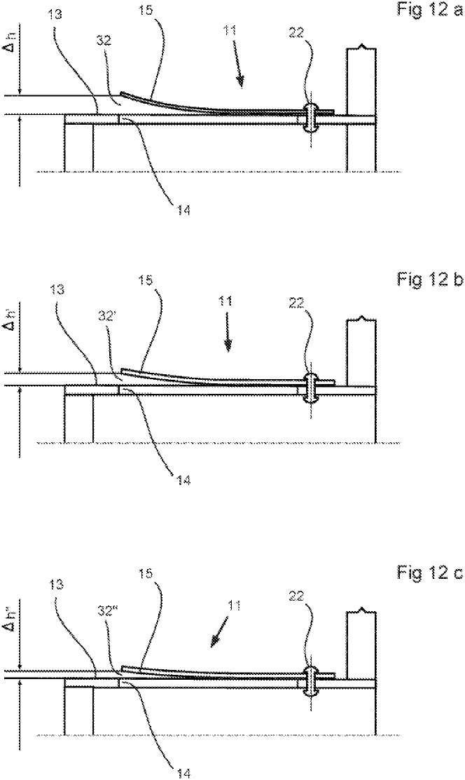

This oscillation behavior of the tongues 15 is shown and elucidated in FIGS. 12a, 12b, and 12c. Firstly, the deflections of the tongues 15 are differentiated in that the distances Δh between the bottom side of the tongue 15 and the top side of the retaining body 13 have different magnitudes. This change in distance is depicted by reference numerals 32, 32′, and 32″. These changes in distance 32, 32′, or 32″ are a function, firstly, of the material used for the tongues 15, which are made of a flexible metal. The oscillation behavior of the tongue 15 changes with different material properties. Furthermore, the oscillation behavior of the tongues 15 may also be influenced by the geometry of the openings 14 and the thickness of the tongues 15.

It has also been shown that for certain pairings of materials, geometries, and pressure conditions in the through channel 4 or in the hollow body 13, an acoustically perceivable tone sequence results due to the oscillation behavior of the tongues 15. These acoustics are used to allow the patient 2 to precisely determine whether his/her breathing behavior meets the desired training or therapeutic purpose. Namely, when the resulting acoustics are weak, or discordant tones are produced, the particular patient 2 recognizes from the prevailing acoustics that the breathing behavior is not correct, and therefore changes must be made to the stenosis 18 or the retaining body 13 or tongues 15 that are used.

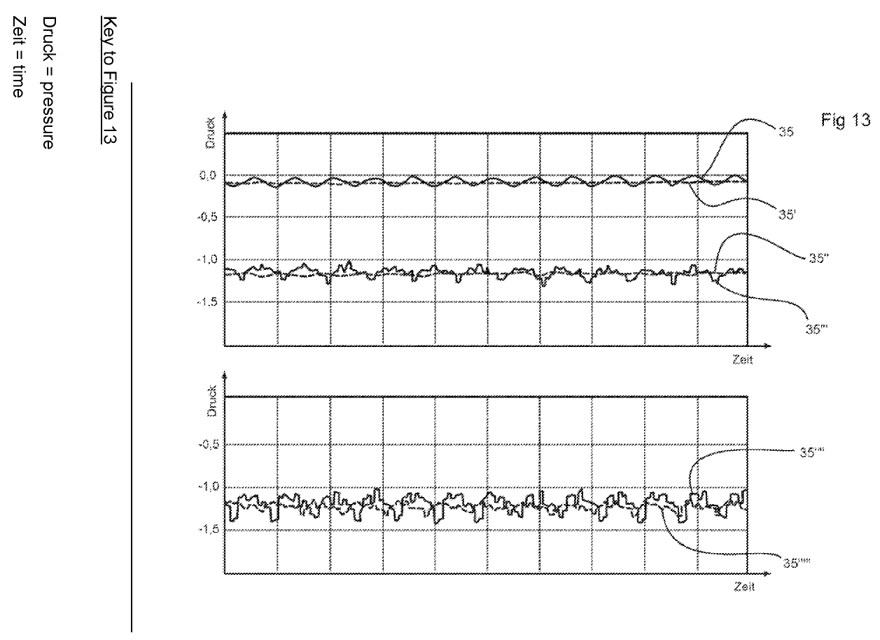

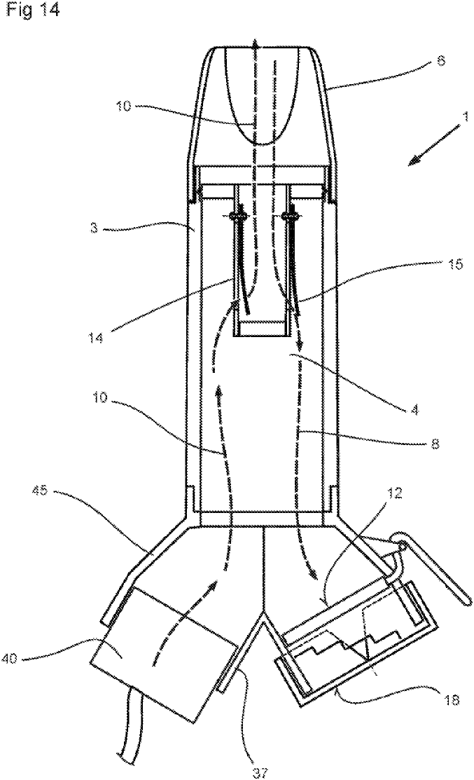

FIG. 13 schematically depicts three different oscillation behaviors. The acoustic behavior of the respiratory therapy device 1 is thus mapped by the predefined internal pressure in the through channel 4 and the flow through the openings 14, which influence and specify the oscillation behavior of the tongues 15. According to FIGS. 12a, 12b, and 12c, the respective distance 32, 32′, or 32″ changes, resulting in the flow rate of the inhaled ambient air 7 or the respiratory air 8. Accordingly, the larger this distance 32, 32′, or 32″, the greater the air volume that flows through the openings 14, or a positive or negative pressure in the through channel 4 is generated or is necessary to set the tongue 15 into oscillation. In combination with the particular stenosis 18 connected upstream, the minimum flow necessary for tone formation or a negative or positive pressure may be precisely set.

The graphs depicted in the diagrams in FIG. 13 show the relationships between these setting options. Time is plotted on the abscissa, and the particular pressure is plotted on the coordinate, where 1 V corresponds to a pressure of 20 cm H₂O. The measurements shown here by way of example have been implemented using a negative pressure. However, this applies in the same way for the positive pressure which results during exhalation. For these measurements with a small clearance distance 32 and the largest or widest cross-sectional area of the through opening 20 of the stenosis 18, a pronounced oscillation in the vibration behavior of the tongues 15 was observed, which has considerable pressure fluctuations even at low pressures. At high pressures these pressure fluctuations are significantly more pronounced, and also range over a high pressure level from -1 V. However, if the smallest cross-sectional area of the through opening 20 at the stenosis 18 is selected or set, the oscillation behavior does not change, and is specified by the dimensions and the stiffness of the material used for the tongues 15.

The combination of various clearance distances 32, 32′, or 32″ for forming the negative and/or positive pressures during inhalation or exhalation, and the stiffness and length of the tongues 15 with the set cross-sectional area of the through opening 20 of the particular stenosis 18, thus precisely defines the necessary pressure required at the start of an oscillation, and accordingly is used for training the respiratory musculature and for clearing bronchial secretions. This oscillation behavior is also acoustically perceived not only by the patient 2, but also by the sensors 23 or 24 mounted at the tongues 15. This selection of the setting options may thus be individually established for each patient 2 in order to optimally influence the therapeutically required parameters

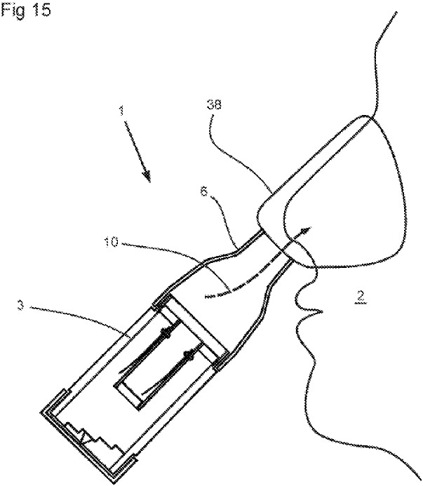

FIG. 14 depicts a structural refinement of the respiratory therapy device according to FIGS. 9 and 10. It is apparent that an adapter 37 is inserted into the free end of the hollow body 13 as a switch for the inhaled ambient air 10 and the respiratory air 8. The adapter 37 has a Y shape, so that it has two openings that face the surroundings and into which one of the stenoses 18 may be respectively inserted. As a result, the pressure state in the through channel 4 may also be set differently for the inhalation process or exhalation process by selecting or adjusting smaller or larger cross-sectional areas of through openings 20 at the particular stenosis 18.

FIG. 15 shows the application of the respiratory therapy device 1 for nasal use. The mouthpiece 6 of the respiratory therapy device opens into a mask 38, which is placed on the outer side of the nose of the patient 20 in a conventional and known manner. The patient 20 may thus also use the respiratory therapy device 1 according to the invention for clearing mucous inside the nasal cavity.

The tongues 15 may be made of a flexible material. It is critical that the tongues 15 vibrate and thus generate the acoustically perceivable vibration of their oscillation. Accordingly, the material used may be made of metal, a hard plastic, or a woven fabric made of these materials.

1. A respiratory therapy device (1) for treating the musculature of respiratory tracts, lung functions, and/or mucous deposits in the lungs, nose, and/or throat of a patient (2),
made up of:
a hollow body (3) in which at least one through channel (4, 5) is provided,
a mouthpiece or nosepiece (6) that is inserted into the hollow body (3) and connected to the patient (2) over the duration of the treatment by the respiratory therapy device (1), and
at least one oscillating body (11, 12) that is situated in the interior of the hollow body (3) and that is set into oscillating vibration by the inhalation and/or exhalation of the patient (2),
characterized in that a retaining body (13) into which at least one opening (14) is incorporated is inserted into the hollow body (3), a freely oscillating tongue (15) is fastened to the retaining body (13) in the region of the opening (14), the opening (14) of the retaining body (13) being closed or opened up in areas via the tongue as a function of the air flows (7, 8) prevailing in the interior of the hollow body (3), the end face (9) of the hollow body (3) opposite from the mouthpiece (6) is open, a stenosis (18) is inserted into this end face (9), at least two through openings (20) being provided in the shell surface (19) of the stenosis, through which the air flows (7, 8) flow into, or conversely, flow out of, the interior of the hollow body (3), the through openings (20) of the stenosis (18) have different cross-sectional areas than air passages for the air flows (7, 8), and the stenosis (18) is rotatably supported in the hollow body (3) in such a way that one of the through openings (20) is selectable or adjustable as an air passage by rotating the stenosis (18).

2. The respiratory therapy device according to claim 1, characterized in that the stenosis (18) has a wall (21) via which the access into the hollow body (3) in the direction of its axis of symmetry (3′) is closed, and the outer contour of the stenosis (18) is adapted to the inner contour of the hollow body (3) .

3. The respiratory therapy device according to claim 1, characterized in that the through openings (20) are closable by means of a plug.

4. The respiratory therapy device according to claim 1, characterized in that the tongue (15) rests on or lifts up from one side of the surface of the hollow body (13), or the tongue (15) allows air to flow in and/or flow out through the opening (14) in an oscillating manner.

5. The respiratory therapy device according to claim 1, characterized in that the first and second oscillating bodies (11, 12) are situated in a breathing direction or in the direction opposite from the breathing direction.

6. The respiratory therapy device according to claim 1, characterized in that the hollow body (13) is designed as a box profile that cuboidally encloses a cavity, a sensor (24) and/or a microphone (25) are/is connected to at least one of the oscillating bodies (11, 12), as the result of which acoustic or electrical signals are transmitted to an external device (26), the oscillating bodies (11, 12) are used as an acoustically perceivable sounding body for checking the correct or desired application of the respiratory therapy device (1), and the openings are provided in an end face of the housing that extends in parallel in the valves.

7. The respiratory therapy device according to claim 1, characterized in that the tongue (15) is made of a flexible material, preferably metal, hard plastic, or a woven fabric made of these materials.
